(19) United States
(12) Patent Application Publication
(10) Pub. No.: US 2015/0087929 A1
(43) Pub. Date: Mar. 26, 2015

(54) Method and System for Population Level Determination of Maximal Aerobic Capacity

(71) Applicant: Tuyymi Technologies LLC, Wilmington, DE (US)

(72) Inventors: Benjamin I. RAPOPORT, New York, NY (US); Craig H. MERMEL, San Jose, CA (US)

(21) Appl. No.: 14/493,178

(22) Filed: Sep. 22, 2014

Related U.S. Application Data
(60) Provisional application No. 61/934,986, filed on Feb. 3, 2014.
(60) Provisional application No. 61/880,528, filed on Sep. 20, 2013.

(51) Int. Cl. A61B 5/00; A61B 5/0205; A61B 5/11

(52) U.S. Cl. CPC A61B 5/7278; A61B 5/1112; A61B 5/0205; A61B 5/024
USPC 600/301; 600/531; 600/484

(57) ABSTRACT

A computerized method for determining maximal oxygen uptake for a user with incomplete data with data collected from a plurality of other users with complete data. The maximal oxygen uptake can be determined by computing similarity metrics between an incomplete data set of self-reported and measured data and complete user data sets, and using a weighted sum of the similarity metrics. The results of the maximal oxygen update calculation can be cross-validated with known user data sets.

### CROSS-REFERENCE TO RELATED APPLICATIONS

This application claims priority under 35 U.S.C. §119(e) to U.S. Provisional Application No. 61/880,528, entitled “Method for Determining Aerobic Capacity”, filed Sep. 20, 2013, the contents of which are incorporated by reference herein.

This application claims priority under 35 U.S.C. §119(e) to U.S. Provisional Application No. 61/934,986 entitled “Method and System for Population-Level Determination of Maximal Aerobic Capacity”, filed Feb. 3, 2014, the contents of which are incorporated by reference herein.

This application is related to U.S. application Ser. No. 14/145,042, entitled “Method for Determining Aerobic Capacity”, filed Dec. 31, 2013, the contents of which are incorporated by reference herein.

All cited references are incorporated herein in their entirety.

### BACKGROUND

The ability of the body to deliver oxygen to its vital organs and tissues, and the ability of those organs and tissues to consume oxygen in the processes of oxidative cellular metabolism, are fundamental to sustaining life in humans and many other species.

At a macroscopic scale, the delivery of oxygen to organs and tissues of the body relies on the lungs, the heart and blood vessels (together comprising the cardiovascular system) and on the blood itself. The heart pumps blood through the lungs, where blood absorbs oxygen. Oxygen-rich blood then returns to the heart, from which it is pumped through the blood vessels that distribute it to the organs and tissues of the body. Tissues absorb oxygen carried by the blood and use the oxygen in the chemical reactions of oxidative metabolism (also known as “aerobic metabolism”), which provide energy for many essential biological functions.

The rate at which a body consumes oxygen at a given point in time is referred to in the art as the \dot{V}O₂, where the symbol V refers to volume and the dot above the V signifies a rate of change with respect to time, so that the symbol \dot{V}O₂ therefore refers to a volumetric flow of oxygen into the tissues of the body. (Gas volumes are typically assumed to be measured at standard temperature and pressure, so that gas volume can be taken to specify a precise molar quantity.) The quantity \dot{V}O₂ is thus a well defined quantity; in the art this quantity is referred to by a variety of terms under various circumstances. In the present disclosure, it will primarily be referred to as “oxygen uptake.”

As a numeric quantity, \dot{V}O₂ measures the overall rate at which the body is engaged in oxidative metabolism.

Since power refers to a rate of energy expenditure, the rate of oxygen consumption, which is directly related to the rate of oxidative metabolic energy expended in aggregate by the cells of the body, is related directly to the aerobic power output of the body. In the interest of controlling for differences in body size, \dot{V}O₂ is typically reported for a given individual in terms of oxygen volume (at conditions of standard temperature and pressure) per unit time per unit body mass (as in milliliters of oxygen per kilogram body mass per minute). The magnitude of the aerobic power output depends not only on the status of the blood and cardiovascular system, but also on the current demands of the body itself and its systems for energy, which may differ greatly, for example, between states of sleep and vigorous exercise.

In assessing the health or fitness of a given individual, from the perspectives of metabolism (energy production) and cardiovascular status, \dot{V}O₂ must therefore be interpreted with respect to any activity being performed by the body. On the other hand, the maximum \dot{V}O₂ achievable by a given individual is, in principle, dependent only on the metabolic and cardiovascular status of that individual. Maximum \dot{V}O₂, which is known in the art by a variety of names (including “aerobic capacity”), is thus of considerable practical use in the assessment of cardiovascular and metabolic health and fitness. In particular, from the standpoint of health and medicine, exercise capacity as quantified by maximum \dot{V}O₂ has been validated as among the most powerful predictors of mortality associated with cardiovascular disease. Myers, J., et al., Exercise Capacity and Mortality among Men Referred for Exercise Testing, New England Journal of Medicine Vol. 346, pp. 793-801 (2002); Earnest, C. P., et al., Maximal Estimated Cardiorespiratory Fitness, Cardiometabolic Risk Factors, and Metabolic Syndrome in the Aerobics Center Longitudinal Study, Mayo Clinic Proceedings, Vol. 88(3), pp. 259-270 (2013); Lavie, et al., Impact of Cardiorespiratory Fitness on the Obesity Paradox in Patients With Heart Failure, Mayo Clinic Proceedings, Vol. 88(3), pp. 251-258 (2013). From another perspective, maximum \dot{V}O₂ is of interest to competitive athletes and those who advise them, as it is a strong predictor of performance ability in many domains of sport. Brooks, et. al., Exercise Physiology: Human Bioenergetics and its Applications (2004) 4^{th} Ed. 2005; McArdle W. D., et al., Exercise Physiology, Lippincott Williams & Wilkins (2009) 7^{th} Ed. 2010.

Another parameter, the time constant of heart rate recovery after exercise, k, also has been demonstrated to predict cardiovascular fitness. Wang L., et al., Time constant of heart rate recovery after low level exercise as a useful measure of cardiovascular fitness, Conf Proc. IEEE Eng. Med. Biol. Soc, Vol. 1, pp. 1799-802 (2006).

In both medical and athletic settings, maximum \dot{V}O₂ is traditionally measured using staged exercise protocols. In schemes such as the widely used Bruce Protocol (Bruce, R. A., et al., Exercising Testing in Adult Normal Subjects and Cardiac Patients, Pediatrics, Vol. 31(4), pp. 742-756 (1963); Bruce, R. A., et al., Maximal Oxygen Intake and Nomographic Assessment of Functional Aerobic Impairment in Cardiovascular Disease, American Heart Journal, Vol. 85(4), pp. 546-562 (1973)), for example, cardiac function may be monitored using electrocardiography, and respiratory volumes as well as oxygen and carbon dioxide gas exchanges may be monitored using clinical spirometry. While such physiologic parameters are measured, an individual patient or athlete is monitored while engaged in standardized forms of exercise (such as treadmill walking or running, or cycle ergometry) at intensities that may be increased in controlled fashion by varying speed, incline, resistance, or other parameters, in a stepwise fashion and at predetermined intervals, until the subject is unable to tolerate further increments in intensity. The point of exhaustion or termination of the test is typically considered the point at which maximum \dot{V}O₂ has been reached, and the corresponding rate of oxygen consumption, determined by clinical spirometry, is then identified as the maximum \dot{V}O₂.

A variety of “sub-maximal” protocols for estimating maximum \dot{V}O₂ have also been described, in which testing stops short of the exhaustion point, and extrapolation methods are used to estimate maximum \dot{V}O₂ on the basis of physiologic data obtained at exercise intensities below that which would elicit exhaustion or maximal oxygen uptake. Observed heart rate and predicted maximum heart rate are common surrogate parameters used in such submaximal protocols. McArdle, W. D., et al., Exercise Physiology, Lippincott Williams & Wilkins (2010).

It will be clear to those skilled in the art how estimates of maximal oxygen uptake can be used in combination with measurements of exercise intensity and duration to estimate other metabolic quantities of interest, including fat and carbohydrate metabolism, lactate production, and water and electrolyte loss during exercise. Brooks, et. al., Exercise Physiology: Human Bioenergetics and its Applications (2004); Rapoport, B. I., Metabolic Factors Limiting Performance in Marathon Runners, Public Library of Science Computational Biology, Vol. 6(10), e1000960 (2010).

The state of the art includes some systems and methods for assessing cardiovascular and aerobic fitness during “free,” unconstrained modes of exercise, as disclosed, for example, by Seppanen and colleagues. Seppanen, et al., Fitness Test, U.S. Pat. Pub. No. 2011-0040193 (2008). However, such systems are unable to account for important physiologic dynamics, and require component methods for eliminating physiologic data captured during periods of non-steady-state physical activity; as such, they do not differ fundamentally from traditional, fixed-protocol physiologic assessments involving assessments through a sequence of physiologic plateaus. The present disclosure describes systems and methods that use mathematical models of physiologic dynamics to enable determination and tracking of aerobic capacity and related physiologic parameters from data continuously acquired during natural activities.

Maximal oxygen uptake is a fundamental indicator of cardiovascular function in both health and disease, of interest to athletes and recreational exercisers as a measure of cardiovascular fitness, and to medical professionals and patients as a predictor of morbidity and mortality from cardiac causes. Existing methods of determining maximal oxygen uptake rely on contrived, fixed, laboratory-based, stepwise exercise protocols; they are time- and resource-intensive, and thus impractical to administer serially to monitor progress; and they typically do not perfectly simulate the natural activities they are designed to reflect.

### SUMMARY

The present disclosure provides methods and systems for determining maximal oxygen uptake for a user with incomplete data with data collected from a plurality of other users with complete data. The methods and systems of the present disclosure include electronically receiving, from at least one user device corresponding to at least one of the complete data users, a plurality of data comprising: a combination of self-reported and measured data sufficient to perform a maximal oxygen uptake calculation for the at least one complete data user, and a maximal oxygen uptake corresponding to the at least one complete data user maximal oxygen uptake calculation; electronically receiving, from a user device corresponding to the incomplete data user, incomplete user data including a subset of the combination of self-reported and measured data received from the at least one complete data user device, the subset of data insufficient to perform a maximal oxygen uptake calculation equivalent to the maximal oxygen uptake calculation corresponding to the at least one complete data user; determining, using a computing device, at least one similarity metric between the incomplete data user combination of self-reported and measured data and the at least one complete data user combination of self-reported and measured data, the at least one similarity metric based on types of data in common between the incomplete data user and the at least one complete user; and estimating, using the computing device, the maximum oxygen uptake of the incomplete data user using a weighted sum of the at least one similarity metric.

In some embodiments, a cross-validation procedure can be used to compute the statistical confidence of the at least one complete data user maximal oxygen uptake. In some embodiments, the cross-validation procedure includes: for each complete data user, determining, using the computing device, a similarity metric between each of the complete data user combination of self-reported and measured data and the other complete data user combination of self-reported and measured data, the similarity metric based on types of data in common between each of the complete data user and the other complete data users; estimating at least one maximum oxygen uptake for each complete data user using a weighted sum of the similarity metrics; determining, for each complete data user, a difference between the estimated maximum oxygen uptake and the calculated maximum oxygen uptake; and using the differences to compute, for each complete data user, a statistical confidence of the estimated complete data user maximal oxygen uptake.

In some embodiments, the user device corresponding to the at least one complete data users comprises a sensor including at least one of a heart rate monitor, a global positioning system (GPS) transponder, and an accelerometer. In some embodiments, the user device corresponding to the incomplete data user comprises a sensor including at least one of a heart rate monitor, a global positioning system (GPS) transponder, and an accelerometer. In some embodiments, the at least one similarity metric is determined using a similarity function. In some embodiments, the similarity function comprises at least one of determining the absolute value between the at least one complete user data and the incomplete user data, determining a Pearson correlation between the at least one complete user data and the incomplete user data, and determining a Euclidean distance between the at least one complete user data and the incomplete user data. In some embodiments, the at least one complete data user combination of self-reported and measured data comprises raw data streams, demographic and biometric parameters, and metrics computed from the raw data and demographic and biometric parameters. In some embodiments, the raw data streams comprise time-stamped series of heart-rate data, motion, and velocity data. In some embodiments, the demographic and biometric parameters comprise age, gender, weight, and height. In some embodiments, the metrics computed from the raw data and demographic and biometric parameters comprise average speed, fastest speed, and total distance traveled each week.

In some embodiments, calculating the maximal oxygen uptake corresponding to the at least one complete data user comprises: (a) electronically measuring instantaneous heart rate data, instantaneous biomechanical data, and instantaneous geophysical data of the user over a period of time, using one or more sensors; (b) setting an oxygen uptake model for the at least one complete data user and storing the oxygen uptake model in memory of a computer; (c) determining, using the computer, a maximum heart rate of the at least one complete data user and storing the maximum heart rate in memory; (d) determining, using the computer, a plurality of instantaneous oxygen uptake estimates over the period of time based in part on user data including the maximum heart rate, the instantaneous biomechanical data, and the instantaneous geophysical data, wherein the at least one complete user data is selected and related to the plurality of instantaneous oxygen uptake estimates using the oxygen uptake model; (e) evaluating, using the computer, a relationship between a real-time heart rate relaxation constant and a real-time maximal oxygen uptake of the at least one complete data user based at least in part on the plurality of the instantaneous oxygen uptake estimates, the maximum heart rate, the instantaneous heart rate data, the instantaneous biomechanical data, and the instantaneous geophysical data, wherein the heart rate relaxation constant comprises a numerical parameter that measures a rate at which the heart rate of a user changes in response to oxygen demand; and (f) determining, using the computer, a maximal oxygen uptake for the at least one complete data user during the aerobic activity, using the relationship between the real-time heart rate relaxation constant and the real-time maximal oxygen uptake.

### BRIEF DESCRIPTION OF THE DRAWINGS

FIG. 1 is a block diagram overview of a system architecture as applied to a single user, according to some embodiments of the present disclosure.

FIG. 2 is a block diagram overview of a system architecture as applied to multiple users, according to some embodiments of the present disclosure.

FIG. 3 is a block diagram overview of a system architecture as implemented for multiple users for whom incomplete data streams are available, according to some embodiments of the present disclosure.

FIG. 4 is block diagram of a system for estimating maximal aerobic capacity in the case of incomplete data, according to some embodiments of the present disclosure.

FIG. 5 is a flowchart illustrating a method by which the system computes the statistical confidence of the estimated maximal aerobic capacities using a cross-validation procedure, according to some embodiments of the present disclosure.

### DESCRIPTION

In the present disclosure, a system for accurately estimating maximal aerobic capacity values in the setting of incomplete or missing data is described. The system takes advantage of the availability of specific data streams, biometric and demographic parameters, and maximal aerobic capacity values measured over a large population of users to estimate maximal aerobic capacity values for users that are missing one or more key data points. U.S. patent application Ser. No. 14/145,042, the entire contents of which are incorporated by reference herein, describes a method for dynamically estimating the maximal oxygen uptake over a large populations of individuals. In the general case, an estimate of maximal oxygen uptake is a function of a set of time series such as heart rate data, biometric data, biomechanical data, and geophysical data, and a set of demographic parameters. Ideally, when estimating maximal oxygen uptake for a given individual, all parameters and data streams are available. In practice, however, some data will not be available for every user. In a large population of users, missing data from individual users can be imputed using statistical methods, based on inference from data obtained from similar users in the population. This disclosure describes such a population-based inference scheme for obtaining and cross-validating maximal oxygen uptake estimates for users with incomplete data sets.

While the statistical approaches described in this disclosure may not fully approach the accuracy of estimates obtained by direct measurement of the key data points, the system has two key advantages over existing methods:

1. By relaxing the input requirements, the system enables maximal aerobic capacity estimates in a much larger population of users and with less user-effort than is possible using existing methods.

2. The accuracy of the maximal aerobic capacity estimate for a given user improves both as the amount of data collected on that user increases, and as the population of users for whom aerobic capacity estimates are available grows.

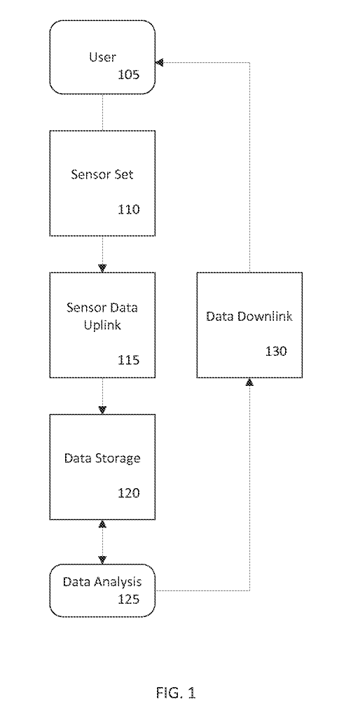

Turning to the drawings, FIG. 1 provides an overview of the system architecture as applied to a single user, according to some embodiments of the present disclosure. The system includes a number of sensors 110 that collect information about each user 105 of the system. As described in the attached filing, the sensors most importantly include heart rate monitors, global positioning system (GPS) transponders, and accelerometers. The system is in principle compatible with any type of wearable sensor that tracks these parameters, although use of other types of sensors is envisioned as well.

The sensors 110 in turn transmit the information they collect from each user 105 to a data storage subsystem 120 through a sensor data uplink 115.

A data analysis subsystem 125 has continuous access to the data accumulated in the data storage subsystem 120, and continuously performs computations of maximal oxygen uptake and other derived measures, using data obtained from the sensors mentioned in the previous paragraphs. The results of these computations, including estimates maximal oxygen uptake, are stored in the data analysis subsystem (125) for later use, and some or all results may be returned to the User (105) through a Data Downlink (130).

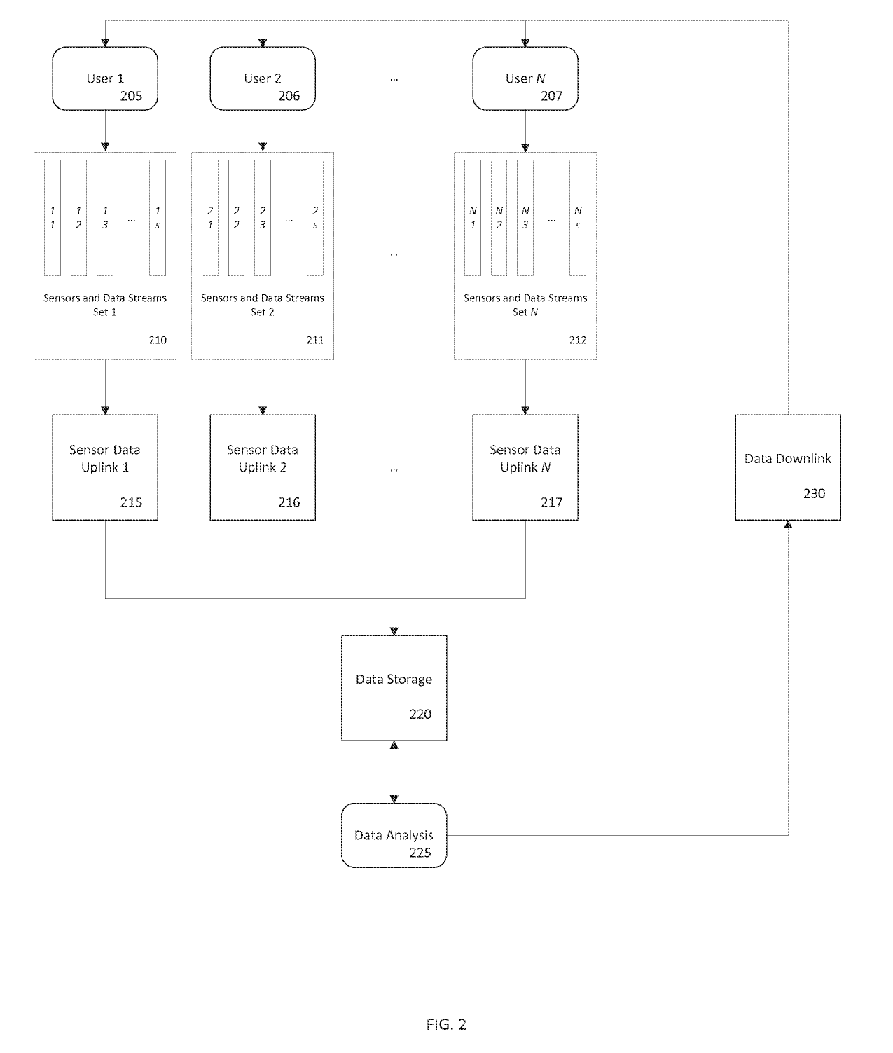

FIG. 2 provides an overview of the system architecture as it may be implemented for multiple users (205, 206, 207, . . . ), according to some embodiments of the present disclosure. As in FIG. 1, which describes the case of a single user 105, multiple users (205, 206, 207, . . . ) are each monitored by corresponding Sets of Sensors and Data Streams (210, 211, 212, . . . ). Each Set of Sensors and Data Streams (210, 211, 212, . . . ) uses a corresponding data uplink (215, 216, 217, . . . ) to transmit information collected from its corresponding user (205, 206, 207, . . . ). This transmission may take place in real time or after a time delay following data collection by the sensors. Data from all data uplinks (215, 216, 217, . . . ) are transmitted to and stored in a central data storage subsystem 220. As in the single-user case described in FIG. 1, a data analysis subsystem 225 has continuous access to the data accumulated in the data storage subsystem 220, and continuously performs computations as diagrammed in the attached disclosure. The results of these computations are stored in the Data Analysis Subsystem 225. In the multi-user case described in FIG. 2, data and computations derived from each user (205, 206, 207, . . . ) are available to the system for use in imputation, as described below. As in the single-user case, some or all results may be returned to the Users (205, 206, 207, . . . ) through a Data Downlink (230).

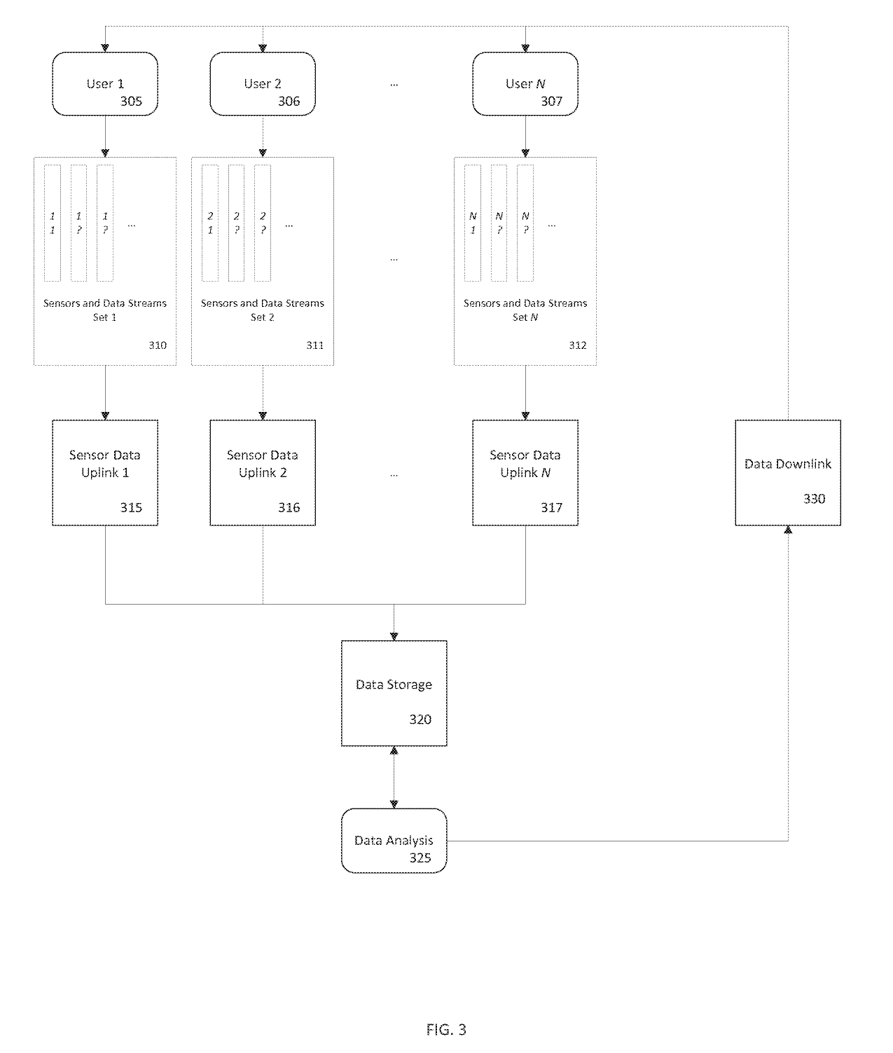

FIG. 3 provides an overview of the system architecture as it may be implemented for multiple users (305, 306, 307, . . . ) for whom incomplete data streams are available, according to some embodiments of the present disclosure. As in FIG. 2, which describes the case of multiple users (205, 206, 207 . . . ) with access to complete sensor sets, these users may or may not be be monitored by one or more Sets of Sensors and Data Streams (310, 311, 312, . . . ) which form a strict subset of the sensors (210, 211, 212) necessary for complete computation of maximal aerobic capacity, as explained in detail in the attached disclosure. Note that the sensor set may not be identical for each of the users (305, 306, 307) so that different numbers and combinations of sensors may be available for the different users. The data that is collected is transmitted through data uplinks (315, 316, 317, . . . ) to the central data storage subsystem 320 where it is stored and available for comparison to other users.

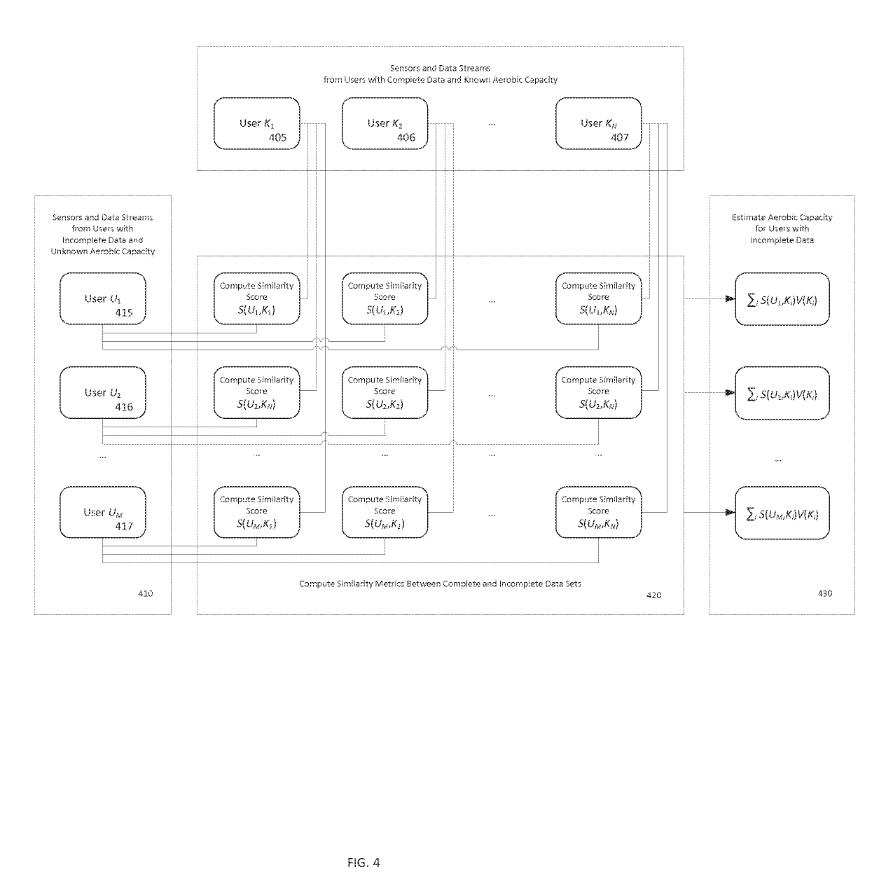

FIG. 4 illustrates a system for estimating maximal aerobic capacity in the case of incomplete data, according to some embodiments of the present disclosure. The system first compares Sensors and Data Streams from Users with Incomplete Data and Unknown Aerobic Capacity (415, 416, 417 . . . , designated with U for “Unknown”) to the population of users (405, 406, 407 . . . ) for whom precise estimates of maximal aerobic capacity are already computed (designated with K for “Known”: “Sensors and Data Streams from Users with Complete Data and Known Aerobic Capacity”). For each such pair of users, the system computes a Similarity Metric, designated by the function S(Uᵢ,Kᵢ) that reflects how closely two users, user U, with incomplete data and user Kⱼ with complete data and known maximal aerobic capacity, matched with respect to the parameters and data streams that are available to the system, including additional metrics derived from the data (collectively, the “similarity metrics”).

The similarity metrics may depend on raw data streams (such as time-stamped series of heart-rate data, motion, and velocity data); demographic and biometric parameters (such as age, gender, weight, and height); or metrics computed from these data streams and parameters (such as average and fastest speed, or the total distance traveled each week). Because in the general case not all sensors and parameters are available for each user, only a subset of all possible similarity metrics can be used to compute the similarity scores involving the user 405.

The specific form of the similarity function will vary according to both the type and nature of the similarity metric. For example, for simple numeric metrics the function may relate to the absolute value of the difference between the metrics, while for time-series data the function may depend on more complex measures of similarity such as the Pearson correlation or Euclidean distance between the data streams, after embedding the data into an appropriate vector space. The overall similarity metric for each pair of users is simply the sum of the outputs of the individual similarity functions applied to all similarity metrics available for the user. In this manner, the system may compute similarity scores for all pairs of users with known VO2max (405, 406, 407 . . . ) against each user with incomplete data (415, 416, 417 . . . ).

The maximal aerobic capacity for each user with incomplete data (415, 416, 417 . . . ) is then estimated as a weighted average (430, “Estimate Aerobic Capacity for Users with Incomplete Data”) of the known maximal aerobic capacities of the users with complete data. In practice some very dissimilar users can be given null (zero) weight so as to simplify the calculation for large sets of users. The weights for this weighted average may be constructed to be identical to or functionally related to the similarity scores, and also to the quality and quantity of data used to make the comparison.

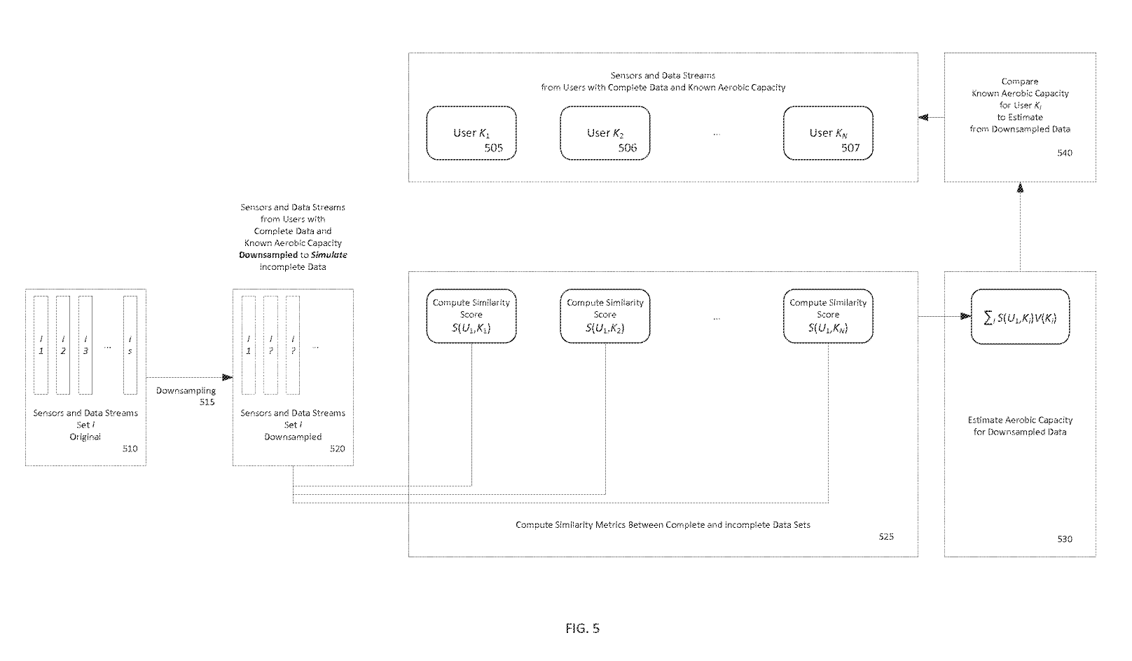

FIG. 5 demonstrates a method by which the system computes the statistical confidence of the estimated maximal aerobic capacities using a cross-validation procedure, according to some embodiments of the present disclosure. Briefly, the system takes a large number of random users with known maximal aerobic capacities (505, 506, 507 . . . ) and down-samples the available complete “Sensor and Data Stream Set” for each user Kᵢ (510) to simulate the incomplete data obtained from a hypothetical user (520) with unknown maximal aerobic capacity. For each down-sampled dataset, the system re-computes the corresponding similarity metrics (525) for each down-sampled user against all other users with complete data (505, 506, 507 . . . ), and then uses the resulting values of the similarity metric to compute the weighted average maximal aerobic capacity estimate 530, as described in the context of FIG. 4. The step “Compare Known Aerobic Capacity for User Kᵢ to Estimate from Downsampled Data” indicates that the difference between the estimated and known maximal aerobic capacities for each of the randomly down-sampled users provides a measure of the error resulting from the estimation process (540). The statistical properties of these differences, computed over many downsampled datasets, provides estimates of statistical confidence of the estimates of aerobic capacity from incomplete data.

Of note, the same cross-validation procedure can be periodically used to increase the accuracy of the weighting procedure 430, by adjusting the function that computes similarity scores 420 so as to minimize the cross-validation error across all users in the database.

To understand the operation of the system more concretely, consider the example of a middle-aged male for whom only basic self-reported demographic and biometric data (age=55, gender=male, weight=200 lbs, height=72 inches, and basic activity level=lightly active) are available. The system has access to a large database of users with known maximal aerobic capacity, from which a population of users with characteristics identical to or highly similar to the current user on all available metrics can be identified. Suppose the system identifies 5 individuals with self-reported characteristics that most closely match the current user, as follows:

| | Individual #1 | | Individual #2 | | Individual #3 | | Individual #4 | | Individual #5 | |
|---|---|---|---|---|---|---|---|---|---|---|
| Age | 55 | | 52 | | 57 | | 54 | | 56 | |
| Gender | Male | | Male | | Male | | Male | | Male | |
| Weight | 195 | lbs | 203 | lbs | 197 | lbs | 195 | lbs | 204 | lbs |
| Height | 71 | inches | 72 | inches | 73 | inches | 71 | inches | 70 | inches |

| | | | | | |
|---|---|---|---|---|---|
| Activity Level | Lightly | Moderately | Lightly | Lightly | Sedentary |
| | active | active | active | active | |
| Maximum | 32.6 | 36.8 | 33.9 | 35.9 | 31.6 |
| Aerobic | | | | | |
| Capacity | | | | | |

Based on these values, the system computes similarity scores and weighting factors between the current user and each of the 5 individuals using the following regression equation:

Similarity Score=0.2*[10−0.1*abs(age1−age2)−5*abs(gender1−gender2)−0.02*abs(weight1−weight2)−0.4*abs(height1−height2)−2.5*abs(activity1−activity2)]

where gender (0=male, 1=female) and activity (0=sedentary, 1=lightly active, 2=moderately active, 3=very active) are numerically encoded.

Thus, the similarity scores for each of these 5 individuals is as follows:

| | Indi- | | Indi- | | | |
|---|---|---|---|---|---|---|
| | vidual | Individual | vidual | Individual | Individual | |
| | #1 | #2 | #3 | #4 | #5 | Sum |
| Similarity | 1.9 | 1.4 | 1.9 | 1.9 | 1.3 | 8.4 |
| Score | | | | | | |

Multiply the similarity score by the maximum aerobic capacity and summing yields:

| | Indi- | | Indi- | | | |
|---|---|---|---|---|---|---|
| | vidual | Individual | vidual | Individual | Individual | |
| | #1 | #2 | #3 | #4 | #5 | Sum |
| Similarity | 61.9 | 52.6 | 63.3 | 67.5 | 41.2 | 286.5 |
| Score × | | | | | | |
| Maximum | | | | | | |
| Aerobic | | | | | | |
| Capacity | | | | | | |

Finally, dividing the weighted sum by the sum of the similarity scores gives an estimated maximum aerobic capacity of 286.5/8.4, or 34.2. By cross-validation (described in greater detail above), it is determined that this result is accurate to within +/−4%, or 1.4, giving an estimated maximum aerobic capacity range of 32.8-35.6.

Cross-validation can refer to the process of performing the same computation described in detail for a new user (for whom the “maximum aerobic capacity” is truly unknown), for each of the users in the system whose “maximum aerobic capacity” is known. In other words, the system can take every user with a known “maximum aerobic capacity,” hide the known value from the system, and estimate the “maximum aerobic capacity” according to some of the embodiments described in detail in the disclosure. The estimated value can be compared to the known value, and the error in the estimation is calculated. Once this is done for every user with complete data, the estimation errors are averaged. In the example provided above, the average estimation error after performing cross-validation for Individuals 1, 2, 3, 4, and 5 is 4%.

The coefficients in the Similarity Score can be assigned somewhat arbitrarily, and many similar functions could potentially be used. In an actual system the coefficients can be tuned through machine learning and iterative cross-validation. For example, the coefficients can be optimized in the Similarity Score by performing the described computation on users for whom all parameters of interest are known, but hiding some of those parameters from the system and asking the system to compute them as though they were unknown. By comparing the values estimated by the system to the actual known values withheld from the system, the coefficients (using machine learning methods known in the art) can be tuned so as to reduce the error between predicted and actual values.

Suppose now that the user records a week's worth of step count data and finds that he takes an average of 7,300 steps per day. The system again queries the database of users and finds the following 5 individuals that best match the current user:

| | Individual #1 | | Individual #2 | | Individual #3 | | Individual #6 | | Individual #1 | |
|---|---|---|---|---|---|---|---|---|---|---|
| Age | 55 | | 52 | | 57 | | 59 | | 51 | |
| Gender | Male | | Male | | Male | | Male | | Male | |
| Weight | 195 | lbs | 203 | lbs | 197 | lbs | 190 | lbs | 214 | lbs |
| Height | 71 | inches | 72 | inches | 73 | inches | 73 | inches | 70 | inches |

| | | | | | |
|---|---|---|---|---|---|
| Activity Level | Lightly | Moderately | Lightly | Moderately | Lightly |
| | active | active | active | active | active |
| Average Daily | 6,100 | 8,600 | 5,900 | 7,200 | 6,900 |
| Step Count | | | | | |
| Maximum | 32.6 | 36.8 | 33.9 | 35.2 | 34.7 |
| Aerobic | | | | | |
| Capacity | | | | | |

Repeating the same process, the system again computes similarity scores, using the following regression equations:

Similarity Score=0.3*[10−0.1*abs(age1−age2)−5*abs(gender1−gender2)−0.02*abs(weight1−weight2)−0.4*abs(height1−height2)−2.5*abs(activity1−activity2)−abs(stepcount1−stepcount2)/2400]

In this case, the coefficient on the similarity score computation has increased from 0.2 to 0.3 due to the fact that adding step-counts increases the ability of the metric to sort the population into users of distinct fitness levels.

Thus, the similarity scores and product of similarity score and maximal aerobic capacity are:

| | Indi- | | Indi- | | | |
|---|---|---|---|---|---|---|
| | vidual | Individual | vidual | Individual | Individual | |
| | #1 | #2 | #3 | #4 | #5 | Sum |
| Similarity | 2.7 | 2.0 | 2.6 | 1.9 | 2.5 | 11.8 |
| Score | | | | | | |
| Similarity | 88.0 | 72.8 | 89.1 | 68.2 | 87.0 | 405.1 |
| Score × | | | | | | |
| Maximum | | | | | | |
| Aerobic | | | | | | |
| Capacity | | | | | | |

which gives an estimated maximum aerobic capacity value of 405.1/11.8, or 34.5. By cross-validation, this result is found to be accurate to within 2.5%, giving a final estimated aerobic capacity of 33.6-35.3, a 40% improvement in confidence over the previous estimate.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

We claim:

1. A computerized method for determining maximal oxygen uptake for a user with incomplete data with data collected from a plurality of other users with complete data, the method comprising:
(a) electronically receiving, from at least one user device corresponding to at least one of the complete data users, a plurality of data comprising:
a combination of self-reported and measured data sufficient to perform a maximal oxygen uptake calculation for the at least one complete data user, and
a maximal oxygen uptake corresponding to the at least one complete data user maximal oxygen uptake calculation;
(b) electronically receiving, from a user device corresponding to the incomplete data user, incomplete user data including a subset of the combination of self-reported and measured data received from the at least one complete data user device, the subset of data insufficient to perform a maximal oxygen uptake calculation equivalent to the maximal oxygen uptake calculation corresponding to the at least one complete data user;
(c) determining, using a computing device, at least one similarity metric between the incomplete data user combination of self-reported and measured data and the at least one complete data user combination of self-reported and measured data, the at least one similarity metric based on types of data in common between the incomplete data user and the at least one complete user; and
(d) estimating, using the computing device, the maximum oxygen uptake of the incomplete data user using a weighted sum of the at least one similarity metric.

2. The computerized method of claim 1, further comprising using a cross-validation procedure to compute the statistical confidence of the at least one complete data user maximal oxygen uptake.

3. The computerized method of claim 2, wherein using a cross-validation procedure includes:
for each complete data user, determining, using the computing device, a similarity metric between each of the complete data user combination of self-reported and measured data and the other complete data user combination of self-reported and measured data, the similarity metric based on types of data in common between each of the complete data user and the other complete data users;
estimating at least one maximum oxygen uptake for each complete data user using a weighted sum of the similarity metrics;
determining, for each complete data user, a difference between the estimated maximum oxygen uptake and the calculated maximum oxygen uptake; and
using the differences to compute, for each complete data user, a statistical confidence of the estimated complete data user maximal oxygen uptake.

4. The method of claim 1, wherein the user device corresponding to the at least one complete data users comprises a sensor including at least one of a heart rate monitor, a global positioning system (GPS) transponder, and an accelerometer.

5. The method of claim 1, wherein the user device corresponding to the incomplete data user comprises a sensor including at least one of a heart rate monitor, a global positioning system (GPS) transponder, and an accelerometer.

6. The method of claim 1, wherein the at least one similarity metric is determined using a similarity function.

7. The method of claim 6, wherein the similarity function comprises at least one of determining the absolute value between the at least one complete user data and the incomplete user data, determining a Pearson correlation between the at least one complete user data and the incomplete user data, and determining a Euclidean distance between the at least one complete user data and the incomplete user data.

8. The method of claim 1, wherein the at least one complete data user combination of self-reported and measured data comprises raw data streams, demographic and biometric parameters, and metrics computed from the raw data and demographic and biometric parameters.

9. The method of claim 8, wherein the raw data streams comprise time-stamped series of heart-rate data, motion, and velocity data.

10. The method of claim 8, wherein the demographic and biometric parameters comprise age, gender, weight, and height.

11. The method of claim 8, wherein the metrics computed from the raw data and demographic and biometric parameters comprise average speed, fastest speed, and total distance traveled each week.

12. The method of claim 1, wherein calculating the maximal oxygen uptake corresponding to the at least one complete data user comprises:
(a) electronically receiving instantaneous heart rate data, instantaneous biomechanical data, and instantaneous geophysical data of the user over a period of time, from the at least one complete data user device;
(b) setting an oxygen uptake model for the at least one complete data user and storing the oxygen uptake model in memory of a computer;
(c) determining, using the computer, a maximum heart rate of the at least one complete data user and storing the maximum heart rate in memory;
(d) determining, using the computer, a plurality of instantaneous oxygen uptake estimates over the period of time based in part on user data including the maximum heart rate, the instantaneous biomechanical data, and the instantaneous geophysical data, wherein the at least one complete user data is selected and related to the plurality of instantaneous oxygen uptake estimates using the oxygen uptake model;
(e) evaluating, using the computer, a relationship between a real-time heart rate relaxation constant and a real-time maximal oxygen uptake of the at least one complete data user based at least in part on the plurality of the instantaneous oxygen uptake estimates, the maximum heart rate, the instantaneous heart rate data, the instantaneous biomechanical data, and the instantaneous geophysical data, wherein the heart rate relaxation constant comprises a numerical parameter that measures a rate at which the heart rate of a user changes in response to oxygen demand; and
(f) determining, using the computer, a maximal oxygen uptake for the at least one complete data user during the aerobic activity, using the relationship between the real-time heart rate relaxation constant and the real-time maximal oxygen uptake.

13. A system configured to determine maximal oxygen uptake for a user with incomplete data with data collected from a plurality of other users with complete data, the system comprising:
(a) a data storage system configured to electronically receive from at least one user device corresponding to at least one of the complete data users, a plurality of data comprising:
a combination of self-reported and measured data sufficient to perform a maximal oxygen uptake calculation for the at least one complete data user, and
a maximal oxygen uptake corresponding to the at least one complete data user maximal oxygen uptake calculation;
(b) the data storage system further configured to electronically receive, from a user device corresponding to the incomplete data user, incomplete user data including a subset of the combination of self-reported and measured data received from the at least one complete data user device, the subset of data insufficient to perform a maximal oxygen uptake calculation equivalent to the maximal oxygen uptake calculation corresponding to the at least one complete data user;
(c) a data analysis subsystem configured to determine at least one similarity metric between the incomplete data user combination of self-reported and measured data and the at least one complete data user combination of self-reported and measured data, the at least one similarity metric based on types of data in common between the incomplete data user and the at least one complete user; and
(d) the data analysis subsystem further configured to estimate the maximum oxygen uptake of the incomplete data user using a weighted sum of the at least one similarity metric.

14. The system of claim 13, wherein the data analysis subsystem is further configured to use a cross-validation procedure to compute the statistical confidence of the at least one complete data user maximal oxygen uptake.

15. The system of claim 14, wherein the data analysis subsystem, as part of the cross-validation feature, is further configured to:
determine for each complete data user a similarity metric between each of the complete data user combination of self-reported and measured data and the other complete data user combination of self-reported and measured data, the similarity metric based on types of data in common between each of the complete data user and the other complete data users;
estimate at least one maximum oxygen uptake for each complete data user using a weighted sum of the similarity metrics;
determine, for each complete data user, a difference between the estimated maximum oxygen uptake and the calculated maximum oxygen uptake; and
use the differences to compute, for each complete data user, a statistical confidence of the estimated complete data user maximal oxygen uptake.

16. The system of claim 13, wherein the user device corresponding to the at least one complete data users comprises a sensor including at least one of a heart rate monitor, a global positioning system (GPS) transponder, and an accelerometer.

17. The system of claim 13, wherein the user device corresponding to the incomplete data user comprises a sensor including at least one of a heart rate monitor, a global positioning system (GPS) transponder, and an accelerometer.

18. The system of claim 13, wherein the data analysis subsystem is further configured to determine at least one similarity metric using a similarity function.

19. The system of claim 18, wherein the similarity function comprises at least one of determining the absolute value between the at least one complete user data and the incomplete user data, determining a Pearson correlation between the at least one complete user data and the incomplete user data, and determining a Euclidean distance between the at least one complete user data and the incomplete user data.

20. The system of claim 13, wherein the at least one complete data user combination of self-reported and measured data comprises raw data streams, demographic and biometric parameters, and metrics computed from the raw data and demographic and biometric parameters.

21. The system of claim 20, wherein the raw data streams comprise time-stamped series of heart-rate data, motion, and velocity data.

22. The system of claim 20, wherein the demographic and biometric parameters comprise age, gender, weight, and height.

23. The system of claim 20, wherein the metrics computed from the raw data and demographic and biometric parameters comprise average speed, fastest speed, and total distance traveled each week.

24. The system of claim 13, wherein, to calculate the maximal oxygen uptake corresponding to the at least one complete data user, the data analysis subsystem is further configured to:
(a) electronically receive instantaneous heart rate data, instantaneous biomechanical data, and instantaneous geophysical data of the user over a period of time;
(b) set an oxygen uptake model for the at least one complete data user and storing the oxygen uptake model;
(c) determine a maximum heart rate of the at least one complete data user and storing the maximum heart rate in memory;
(d) determine a plurality of instantaneous oxygen uptake estimates over the period of time based in part on user data including the maximum heart rate, the instantaneous biomechanical data, and the instantaneous geophysical data, wherein the at least one complete user data is selected and related to the plurality of instantaneous oxygen uptake estimates using the oxygen uptake model;
(e) evaluate a relationship between a real-time heart rate relaxation constant and a real-time maximal oxygen uptake of the at least one complete data user based at least in part on the plurality of the instantaneous oxygen uptake estimates, the maximum heart rate, the instantaneous heart rate data, the instantaneous biomechanical data, and the instantaneous geophysical data, wherein the heart rate relaxation constant comprises a numerical parameter that measures a rate at which the heart rate of a user changes in response to oxygen demand; and
(f) determine a maximal oxygen uptake for the at least one complete data user during the aerobic activity, using the relationship between the real-time heart rate relaxation constant and the real-time maximal oxygen uptake.
